(19) United States
(12) Patent Application Publication
(10) Pub. No.: US 2011/0034991 A1
(43) Pub. Date: Feb. 10, 2011

(54) ENDOPROSTHESIS AND METHOD FOR PRODUCING SAME

(75) Inventors: Harald Barthel, Erlangen (DE); Matthias Fringes, Ansbach (DE); Bodo Gerold, Zellingen (DE); Johannes Riedmueller, Nuernberg (DE)

Correspondence Address: BARNES & THORNBURG LLP, Suite 1150, 3343 Peachtree Road, N.E., Atlanta, GA, 30326-1428, US

(73) Assignee: BIOTRONIK VI PATENT AG, Baar (CH)

(21) Appl. No.: 12/902,567

(22) Filed: Oct. 12, 2010

Related U.S. Application Data
(62) Division of application No. 11/834,824, filed on Aug. 7, 2007, now abandoned.

(30) Foreign Application Priority Data
Aug. 7, 2006 (DE) 10 2006 038 232.3

(51) Int. Cl. A61F 2/82

(52) U.S. Cl. 623/1.15; 623/1.42

(57) ABSTRACT

An endoprosthesis, in particular, an intraluminal endoprosthesis, e.g., a stent, having a basic mesh and a functional element attached to a carrier structure, the functional element having a different material composition than the material of the basic mesh in at least a portion of its volume. The carrier structure is arranged on the basic mesh in the first essentially finger-shaped end and protrudes away from the mesh essentially like a projection. The functional element is arranged on an area of the carrier structure protruding away from the base body and at least partially surrounds the area of the carrier structure.

### PRIORITY CLAIM

This patent application is a divisional of co-pending U.S. patent application Ser. No. 11/834,824, filed Aug. 7, 2007, which claims priority to German Patent Application No. 10 2006 038 232.3, filed Aug. 7, 2006, the disclosures of which are incorporated herein by reference in their entirety.

### FIELD

The present disclosure relates to an endoprosthesis, in particular, an intraluminal endoprosthesis, e.g., a stent, having a basic mesh and a functional element attached to a carrier structure and having a different material composition in comparison with the material of the basic mesh in at least a portion of its volume. The present disclosure also relates to a method for producing such an endoprosthesis.

### BACKGROUND

Stents are endovascular prostheses which may be used for treatment of stenoses (vascular occlusions). Stents typically have a hollow cylindrical or tubular basic mesh which is open at both of the longitudinal ends of the tubes. The tubular basic mesh of such an endoprosthesis is inserted into the blood vessel to be treated and serves to support the vessel.

Such stents usually have two states, namely, a compressed state having a small diameter and an expanded state having a larger diameter. In the compressed state, the stent can be inserted by means of a catheter into the vessel to be supported and positioned at the location to be treated. At the site of the treatment, the stent is dilated by means of a balloon catheter, for example, or automatically develops into the expanded state (such as when using a shape memory metal as the stent material) due to heating in the blood to a temperature above the transition temperature of the stent material. The basic mesh of the stent is exposed to a high mechanical stress because of this change in diameter.

The basic body of the stent, which is designed as a mesh structure, usually made of a metallic material, a ceramic and/or a plastic, is also exposed to tensile and compressive stresses due to the fact that, after being inserted into the blood vessel, the stent adapts to the shape and movement of the vessel.

It is known that stents may be provided with functional elements having a different material composition in comparison with the material of the basic mesh in at least a portion of their volume. These functional elements serve, for example, to determine the position of a stent in the body or to release medications.

The position of a stent is frequently determined by means of imaging methods, e.g., by means of an x-ray beam device. Since the materials used for the basic mesh of stents usually absorb x-ray radiation only to a slight extent, i.e., they are radiolucent, stents are often provided with x-ray markers which contain a material having a higher absorption of x-rays (radiopaque material).

German Patent Application No. 103 17 241 A1 discloses a stent having a metallic radiolucent basic mesh and marker element having radiopaque material. By cutting out pieces, recesses that act as a carrier structure are provided in webs in the basic mesh of the known stent; marker elements are later welded into these recesses which are then surrounded by a cover layer of silicon carbide. Another option disclosed in this application, although it is somewhat expensive, consists of manufacturing the basic mesh completely from a nitinol wire having a gold core that serves as an x-ray marker. The nitinol wire with a gold core forms the entire end section of the stent and is joined to the basic mesh by welding.

A similar approach is also described in German Patent Application No. 100 64 596 A1, Which discloses a method for applying a marker element to a stent comprising a free-flowing or pourable material or mixture of materials that does not solidify, e.g., in the form of granules, is introduced into a recess in the basic mesh of the stent and is solidified there. Solidification of this material is accomplished by means of sintering, for example.

U.S. Pat. No. 6,174,329 B1 also discloses a coated stent whose basic mesh is radiolucent and which is partially or completely provided with a radiopaque layer. Furthermore, a protective layer which protects the coated stent from scratches or galvanic corrosion and increases the biocompatibility and blood compatibility of the stent may also be provided. In the case of partial coating, the radiopaque materials are applied to the straight sections of the stent and not to the curved portions because the curved sections are exposed to greater mechanical stresses during expansion.

Finally, U.S. Pat. No. 6,293,966 B1 discloses a stent which contains radiopaque marker elements and has C-shaped elements on the distal and/or proximal ends, each forming an essentially spherical receptacle. These receptacles are used for insertion of marker elements with spherical end sections. The spherical end sections are attached in a form-fitting manner, optionally by means of a weld, in the receptacles formed by the C-shaped elements.

With the known stents, there is still the problem that the functional elements which contain the radiopaque materials and are attached directly to the mesh structure of the stent are exposed to stress peaks in the borderline area between the two materials in expansion and adaptation to the vascular geometry because of the difference in material between the basic mesh and the functional element due to the notching effect of the functional element; and, therefore, defects may occur in this area.

In addition, errors may occur in the measurements in determination of the stent position using radiopaque markers as the functional elements because the radiopaque material often has only a slight extent in the plane of image acquisition and perpendicular thereto. Small areas, e.g., currently on the order of 200 μm×200 μm in the plane of acquisition, yield approximately one pixel with a measurement signal with the measurement equipment currently in use. When these signals occur in isolation, they are often eliminated again in machine generation of the image after conclusion of the measurement because such image components are often classified as measurement errors. Therefore, the largest possible extent of the radiopaque material in the plane of acquisition must be achieved, so that several pixels arranged side-by-side have measurement signals. Furthermore, a great extent of the x-ray marker in a direction perpendicular to the plane of acquisition is advantageous, because the absorbed portion of the x-rays is proportional to the thickness of the material through which the x-ray passes. The stent may be oriented in any direction in the body and thus to the plane of acquisition of the image, so it is consequently desirable for the volume filled by the x-ray marker to be as large as possible to avoid measurement errors in determination of the stent position.

The x-ray markers with the known stents mentioned above have only a small volume. With the arrangement of radiopaque material in recesses, the volume of the material is limited by the internal dimensions of the recess. In coating the basic mesh of the stent with radiopaque material, the thickness of these layers is small because with larger layer thicknesses the influence on flow of the liquids, such as blood flowing in the blood vessels through the coated stent sections, is too great; and, therefore, a restenosis may be facilitated.

It is also known that functional elements may be provided on stents containing medications, e.g., having an anti-inflammatory or antiproliferative effect. With regard to these functional elements, it is also desirable for the volume of the functional elements to be chosen to be as large as possible, so that a larger volume of active ingredient can be accommodated and the duration of effect of the medications can be prolonged.

In the case of stents whose basic mesh consists of an absorbable magnesium alloy, there is the additional problem that, with the arrangement of functional elements, e.g., marker elements made of gold or silver on the basic mesh of the stent, contact corrosion can occur in the contact area between the two materials. The contact corrosion leads to destruction of the stent and/or separation of the functional element from the stent structure.

### SUMMARY

The present disclosure describes several exemplary embodiments of the present invention.

One aspect of the present disclosure provides an endoprosthesis, in particular an intraluminal endoprosthesis, e.g., a stent, comprising (a) a basic mesh, and (b) a functional element attached to a carrier structure and having a different material composition in at least a portion of its volume in comparison with the material of the basic mesh, wherein the carrier structure is arranged on the basic mesh on the first essentially finger-shaped end and protrudes away from the basic mesh, and wherein the functional element is arranged on an area of the carrier structure that protrudes away from the base body and at least partially surrounds the area of the carrier structure.

Another aspect of the present disclosure provides a method for producing an endoprosthesis comprising (a) producing an endoprosthesis comprising a basic mesh, and a functional element attached to a carrier structure and having a different material composition in at least a portion of its volume in comparison with the material of the basic mesh, wherein the carrier structure is arranged on the basic mesh on the first essentially finger-shaped end and protrudes away from the basic mesh; wherein the functional element is arranged on an area of the carrier structure that protrudes away from the base body and at least partially surrounds the area of the carrier structure; wherein the base mesh together with the at least one carrier structure is made of a hollow cylinder.

A further aspect of the present disclosure provides a method for producing an endoprosthesis, comprising (a) producing an endoprosthesis comprising a basic mesh, and a functional element attached to a carrier structure and having a different material composition in at least a portion of its volume in comparison with the material of the basic mesh, wherein the carrier structure is arranged on the basic mesh on the first essentially finger-shaped end and protrudes away from the basic mesh; wherein the functional element is arranged on an area of the carrier structure that protrudes away from the base body and at least partially surrounds the area of the carrier structure; wherein the at least one carrier structure is produced separately from the basic mesh and is then attached to the basic mesh.

A feature of the present disclosure is an endoprosthesis with which the functional element has a large volume which should ensure that the functional element is exposed to the lowest possible mechanical stresses so that a defect in the stent in the area of the functional element, in particular, separation of the functional element from the basic mesh of the stent, is prevented. Another feature of the present disclosure is also to provide an inexpensive method for production of such an endoprosthesis.

This feature is achieved by an endoprosthesis whose carrier structure is arranged on a first essentially finger-shaped end, i.e., at one end of the basic mesh, and protrudes away from the basic mesh essentially in the form of an extension, whereby the functional element is arranged on an area of the carrier structure protruding away from the basic mesh and at least partially surrounds the area of the carrier structure.

The functional element is arranged only in a point-shaped area, namely, on the first finger-shaped end of the carrier structure and is attached to the basic mesh so that the functional element can move flexibly on and with the basic mesh, so that in dilation of the basic mesh, for example, there is no plastic deformation, and the stresses transferred from the basic mesh to the carrier structure can be dissipated, for example. Furthermore, the functional element surrounds the carrier structure in an area of the carrier structure which protrudes away from the basic mesh and has preferably a smaller diameter in the area than the structures of the basic mesh so that the volume of the functional element is greater than that in the known art.

In one exemplary embodiment, the functional element completely surrounds or encloses the second end of the carrier structure protruding away from the basic mesh. In this way, an especially large volume of the functional element is achieved; and, furthermore, good anchoring of the functional element on and attachment to the carrier structure are achieved.

Especially with regard to the volume, a droplet, disk or spherical shape forms an advantageous shape for the functional element. Any other geometric shape that is known to those skilled in the art and can be produced, such as an ellipsoid shape, a cubical shape and/or a star shape may also be provided as an alternative. These shapes can be manufactured very inexpensively.

The functional element may consist at least partially of a radiopaque material, so that the position of the stent in the body can be determined easily. For the radiopaque material, preferably one or more of the elements are used from the group consisting of gold, platinum, silver, tungsten, iodine, tantalum, yttrium, niobium, molybdenum, ruthenium, rhodium, barium, lanthanum, cerium, praseodymium, neodymium, samarium, europium, gadolinium, terbium, dysprosium, holmium, erbium, thulium, ytterbium, lutetium, hafnium, rhenium, osmium and bismuth and/or one or more of the radiopaque compounds of these elements and/or barium sulfate, bismuth trioxide, bromine, iodine, iodide, titanium oxide and zirconium oxide.

The functional element especially preferably consists of a marker alloy, which is known from German Patent Application No. 103 61 942. The functional element consists of a biodegradable basic component or basic alloy, in particular, from the group of elements consisting of magnesium, iron, tungsten and/or zinc. In addition, the functional element contains one or more of the aforementioned radiopaque elements and/or one or more radiopaque compounds from these elements.

An especially preferred composition is MgxYby where x=10-60 at % and y=40-90 at % where x and y together, and including any production-related impurities, yield a total of 100 at %. The composition Mg31.5Yb68.5 of the functional element material is also especially preferred.

Alternatively or additionally, at least one functional element or endoprosthesis may be provided with a pharmaceutically active substance.

For purposes of the present disclosure, an “active pharmaceutical substance” is an active ingredient of vegetable, animal or synthetic origin which is used in a suitable dosage as a therapeutic agent for influencing conditions or functions of the body, as a replacement for active ingredients naturally produced by the human or animal body and to eliminate or neutralize disease pathogens or exogenous substances. The release of the substance in the environment of the endoprosthesis has an effect on the course of healing and/or counteracts pathological changes in the tissue due to the surgical procedure.

The pharmaceutically active substances have an anti-inflammatory and/or antiproliferative and/or spasmolytic and/or endothelium-forming effect, so that restenoses, inflammations or vasospasms can be prevented.

Preferred active ingredients, especially for treatment or prevention of in-stent restenosis, which are especially suitable for incorporation into a polymer matrix of an inventive implant, are selected from the group comprising:
lipid regulators (fibrates),
immunosuppressants,
vasodilators (sartane),
calcium channel blockers,
calcineurine inhibitors (tacrolimus),
antiphlogistics (cortisone, diclofenac),
anti-inflammatories (imidazole),
antiallergics,
oligonucleotide (dODN),
estrogens (genistein),
endothelium-forming agents (fibrin),
steroids,
proteins/peptides,
proliferation inhibitors,
analgesics, and
antirheumatics.

Furthermore, the functional element may contain polymers or endogenous substances that function as a matrix material to take up the aforementioned radiopaque materials and/or the aforementioned pharmaceutically active substances. Absorbable (i.e., bioabsorbable or degradable) polymers or nonabsorbable (i.e., permanent or nondegradable) polymers are preferably used as such polymers, especially preferably collagens or cholesterols.

Preferred polymers for the polymer matrix of the inventive implant are selected from the group consisting of:
nonabsorbable/permanent polymers such as:
polypropylene, polyethylene, polyvinyl chloride, polyacrylate (polyethyl acrylate and polymethyl acrylate, polymethyl methacrylate, polymethyl-coethyl acrylate, ethylene/ethyl acrylate), polytetrafluoroethylene (ethylene/chlorotrifluoroethylene copolymer, ethylene/tetrafluoroethylene copolymer), polyamide (polyamideimide, PA-11, PA-12, PA-46, PA-66), polyetherimide, polyether sulfone, poly(iso)butylene, polyvinyl chloride, polyvinyl fluoride, polyvinyl alcohol, polyurethane, polybutylene terephthalate, silicones, polyphosphazenes, polymer foams (from carbonates, styrene, for example) as well as the copolymers and blends of the classes listed and/or the class of thermoplastics and elastomers in general;
absorbable/bioabsorbable/degradable polymers, such as:
polydioxanone, polyglycolide, polycaprolactone, polylactide (poly-L-lactide, poly-D,L-lactide and copolymers and blends such as poly(L-lactide-coglycolide), poly(D,L-lactide-coglycolide), poly(L-lactide-co-D,L-lactide), poly-(L-lactide-cotrimethylene carbonate)), tri-block copolymers, polysaccharides (chitosan, levan, hyaluronic acid, heparin, dextran, cellulose, etc.), polyhydroxyvalerate, ethylvinyl acetate, polyethylene oxide, polyphosphoryl choline, fibrin, albumin, polyhydroxybutyric acid (atactic, isotactic, syndiotactic and blends thereof), and the like.
Especially preferred are the polylactides (poly-L-lactide, poly-D,L-lactide and copolymers and blends such as poly(L-lactide-coglycolide), poly(D,L-lactide coglycolide), poly(L-lactide-co-D,L-lactide), poly(L-lactide-cotrimethylene carbonate)).

In one exemplary embodiment, the carrier structure can be manufactured especially easily and inexpensively if the carrier structure is designed to be essentially finger shaped. In another exemplary embodiment, the carrier structure has a first finger on one end and on its second end has at least one second finger branching off from the first finger. This carrier structure creates the essentially point-shaped flexible connection of the functional element to the basic mesh while creating a structure to which the material of the functional element can be secured well due to the branching at the second end. The carrier structure has a large surface area which serves to anchor the functional element. This approach can be further improved if the carrier structure has a plurality of fractal branches at the second end. Likewise, an advantageously large surface area for anchoring the functional element is offered by the carrier structure if the carrier structure forms tree-like branches at its second end.

The volume taken up by the functional element may be further increased if the carrier structure has a smaller extent than the basic mesh structure in the radial direction to the hollow cylindrical basic mesh, and does so in the area in which the functional element is arranged on the carrier structure.

In another exemplary embodiment, the carrier structure consists at least partially of an electrically insulating material, in particular, plastic and/or ceramic. The electrically insulating material is arranged so that the basic mesh and the functional element are electrically insulated from one another to prevent contact corrosion.

To eliminate the problem of contact corrosion, in another exemplary embodiment, the carrier structure, which may be made inexpensively of the material of the basic mesh in the production of the basic mesh, is provided with an electrically insulating material, e.g., a ceramic and/or a plastic, especially preferably with such an insulating coating, in particular, where the functional element is attached to the carrier structure. Due to the coating with the electrically insulating material, contact between the non-noble magnesium alloy and the more noble radiopaque material is prevented.

According to another exemplary embodiment, a plurality of carrier structures with functional elements containing a pharmaceutically active substance is provided on the endoprosthesis; the functional elements are arranged in a uniform distribution over the entire wall of the mesh structure. This achieves an especially well distributed release of the pharmaceutically active substance in space.

Alternatively or additionally, a plurality of carrier structures with functional elements having radiopaque material is arranged at the distal and/or proximal end of the endoprosthesis, preferably on a circumferential line. It is especially easy to position the stent in this way because the functional elements are attached to a well-defined section of the stent.

The present disclosure also provides a method for producing an endoprosthesis in which the basic mesh is manufactured together with the at least one carrier structure from a hollow cylinder. As a result, the carrier structures may be exposed to higher mechanical loads. Joint production of the basic mesh structures and carrier structures is also inexpensive.

The two- or three-dimensional basic mesh structure and carrier structures are produced by laser beam cutting or water jet cutting or by chemical or electrochemical etching methods, with and without the use of lithographic techniques. Then in one exemplary embodiment, mechanical shaping methods (e.g., pressing) may be performed to reduce the extent of the carrier structure in the radial direction in comparison with the extent of the basic mesh and/or bending, preferably while retaining the extent of the carrier structure in comparison with that of the basic structure.

The present disclosure also provides a method for manufacturing an endoprosthesis in which the at least one carrier structure is manufactured separately from the basic mesh and then is attached to the basic mesh. The carrier structure is preferably provided with the functional element on the basis of a method, as described further below, before attaching the carrier structure to the basic mesh. The two- or three-dimensional carrier structures are first manufactured by laser beam cutting or water jet cutting or by chemical or electrochemical etching methods with or without the use of lithographic techniques or by punching, preferably from a starting material in the form of a sheet. In one exemplary embodiment, the carrier structures may then be processed further and refined by mechanical shaping methods, e.g., pressing or bending. Optionally the carrier structure may be subjected to a sintering step.

The prefabricated carrier structure can be attached to the basic mesh by welding, soldering or gluing. In addition, the carrier structure may also be designed as a fitting pin on the first finger-shaped end, the pin being attached to the basic mesh by a press fit. The carrier structure may also be clipped onto the basic mesh.

The functional elements can be attached to the respective carrier structure, which has been produced separately or jointly with the basic mesh by welding, soldering, gluing or pressing. Likewise, it is possible to attach the functional element to the respective carrier structure by spraying, dipping, dunking or other known coating methods. Furthermore, it is possible to provide a mechanically stable coating which is situated between the carrier structure and the functional element in the case of a functional element designed as a depot for pharmaceutically active substances, or the mechanically stable coating may be designed as the surface of the functional element in the case of a functional element designed as a marker element.

Another method for attaching the functional elements is to embed the radiopaque material and/or the pharmaceutically active substance in a matrix of a carbon polymer or another plastic, preferably from a degradable polymer as defined above, to apply a drop thereof to the respective carrier structure and then solidify the polymer, e.g., by polymerization, preferably using a reagent to initiate the polymerization or by curing, preferably by IR curing or by drying, preferably drying by means of lasers or by means of a pyrolysis process or other conventional methods with which those skilled in the art are familiar. For introducing the radiopaque material or the active ingredient into the matrix, the radiopaque material or the active ingredient is preferably present in a particle size of several micrometers or less, especially preferably as a nanocrystalline material. For applying the drop to the carrier structure, preferably a suitably shaped casting mold can be used. Likewise, it is also possible to proceed if a ceramic material is used as the matrix material in the same way.

In another exemplary embodiment, the radiopaque material and/or the pharmaceutically active substance may be attached to the respective carrier structure by a method such as that described in German Patent Application No. 100 64 596 A1, for example. Molds are preferably also provided for this purpose, the material to be solidified is arranged in the molds in such a way that the functional elements are attached to the respective carrier structure after the conclusion of the manufacturing process.

In the production of the carrier structures, optionally with the functional elements together with the basic mesh from a hollow cylinder, it may also be designed as a sandwich material of at least two different materials in at least some areas. In one exemplary embodiment, for example, a hollow cylinder made of a biodegradable magnesium alloy may be provided with a hollow cylinder having a smaller diameter and made of the material of the carrier structure (and/or optionally a marker alloy) being applied, e.g., by friction welding. The processing by means of the above methods may be accomplished in such a way that the corresponding layer is “left standing”, i.e., the layer is not removed only at those locations where the carrier structures and/or functional elements are to be formed.

### BRIEF DESCRIPTION OF THE DRAWINGS

Additional features, advantages and possible applications of the present disclosure are derived from the following description of exemplary embodiments on the basis of the drawings. All the features described and/or illustrated here may constitute the subject of the disclosure either alone or in any combination, regardless of how they are combined in individual claims or the reference back to them.

FIG. 1a is a schematic view of a detail of a proximal or distal end section of a first exemplary embodiment of a stent shown in a view from the side with partial sectional views through the basic mesh of the stent in the area of the carrier structure, through the carrier structure and through the functional element;

FIG. 1b is a schematic view of a proximal or distal end section of the exemplary embodiment of FIG. 1;

FIG. 2 is a schematic detail view of a proximal or distal end section of another exemplary embodiment of a stent shown in a view from the side with partial sectional views through the basic mesh of the stent in the area of the carrier structure, through the carrier structure and through the functional element;

FIG. 3 is a schematic view of the finger-shaped carrier structures of FIGS. 1a and 1b;

FIG. 4 is a schematic view of a detail of a proximal or distal end section of a further exemplary embodiment of a stent shown in a view from the side with partial sectional views through the basic mesh of the stent in the area of the carrier structure, through the carrier structure and through the functional element;

FIG. 5 is a schematic view of a detail of a proximal or distal end section of an additional exemplary embodiment of a stent shown in a view from the side with partial sectional views through the basic mesh of the stent in the area of the carrier structure, through the carrier structure and through the functional element;

FIG. 6 is a schematic view of a detail of a proximal or distal end section of yet another exemplary embodiment of a stent shown in a view from the side with partial sectional views through the basic mesh of the stent in the area of the carrier structure, through the carrier structure and through the functional element;

FIG. 7 is a schematic view of a longitudinal section through a basic mesh section of an additional exemplary embodiment of a stent having a longitudinal section through a carrier structure and a functional element; and

FIG. 8 is a schematic view of a detail of the stent wall of another exemplary embodiment of a stent in a view from the side.

### DETAILED DESCRIPTION

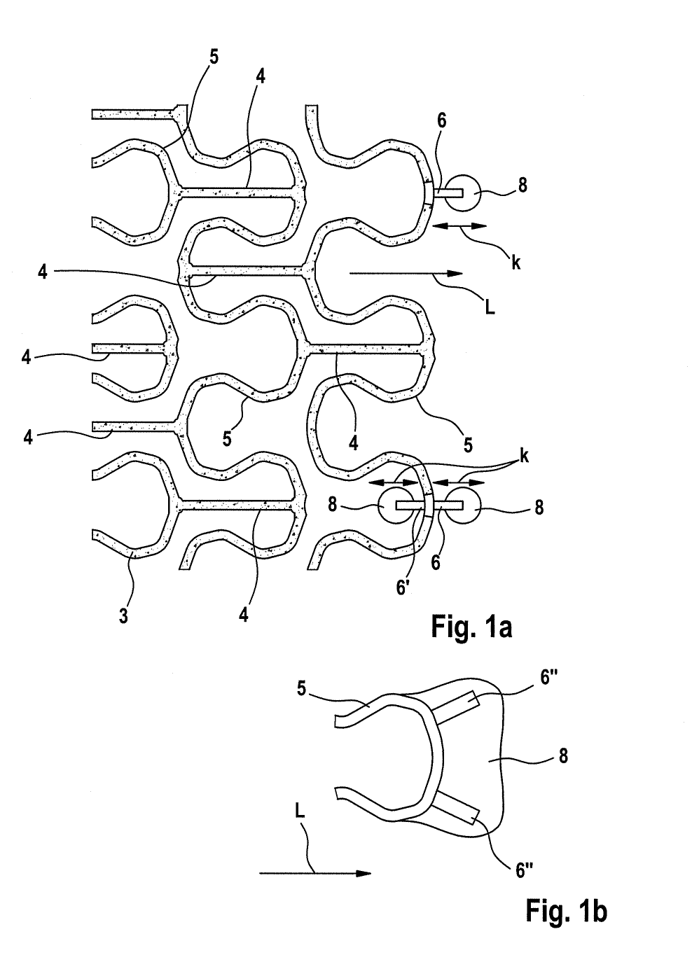

The detail of an end section of the first exemplary embodiment shown in FIG. 1a is a basic mesh 3 with webs 4 running in the longitudinal direction L and zigzag or meandering webs 5. The zigzag or meandering pleated webs 5 are connected to the webs 4 running in the longitudinal direction L and together with the webs 4 form the basic structure of the stent, which is shaped on the whole as tubes running in the longitudinal direction L.

The basic structure of the stent preferably consists of a metallic material consisting of one or more metals from the group consisting of iron, magnesium, nickel, tungsten, titanium, zirconium, niobium, tantalum, zinc, silicon, combinations thereof and the like and optionally a second component from one or more metals from the group consisting of lithium, sodium, potassium, calcium, manganese, iron, tungsten, combinations thereof and the like, preferably a zinc-calcium alloy. In another exemplary embodiment, the basic mesh 3 consists of a memory material comprising one or more materials from the group consisting of nickel-titanium alloys and copper-zinc-aluminum alloys; the basic mesh 3 preferably consists of nitinol. In another preferred exemplary embodiment, the basic mesh 3 of the stent is made of stainless steel or, even more preferably, made of alloy 316L, preferably from a Cr—Ni—Fe steel or a Co—Cr steel. Furthermore, the basic mesh of the stent may be made at least partially of plastic and/or a ceramic.

In another exemplary embodiment the basic mesh consists of an absorbable magnesium alloy having the following composition:
rare earths 2.0 to 30.0 weight-percent,
yttrium 0.0 to 20.0 weight-percent,
zirconium 0.3 to 5 weight-percent,
remainder 0 to 10.0 weight-percent (optionally neodymium),
   whereby magnesium (at least 60.0 weight-percent) constitutes the remainder of the alloy to a total of 100 weight-percent.

The stent basic mesh 3 is produced by first providing a base body in the form of a hollow cylinder (tube) from which the structure of the basic mesh is produced, e.g., by means of a cutting technique, preferably by laser beam cutting or water jet cutting, or by chemical or electrochemical etching methods, with or without the use of lithographic techniques. Then the surface of the stent basic mesh can be machined, especially finished, smoothed and/or polished.

Finger-shaped carrier structures 6 are attached to the zigzag or meandering webs 5 arranged at the farthest point in the direction of the proximal or distal end of the stent illustrated in FIG. 1a. Along a direction k, which indicates the direction of extent of the longitudinal axis of the carrier structure and which runs, in this case, parallel to the longitudinal direction L of the stent, the carrier structures 6 have their greatest extent. The carrier structures 6 may have a cylindrical or cubic shape, for example, so that the cross section perpendicular to the direction k is designed to be essentially rectangular or round. The carrier structures 6 are attached to the web 5 at the first finger-shaped end along the direction k and protrude in the longitudinal direction L away from the basic mesh 3 of the stent such that the second protruding end of the carrier structure 6 is directed away from the basic mesh 3.

By analogy with the carrier structure 6, another finger-shaped carrier structure 6′ is arranged with the finger-shaped end on the zigzag or meandering web 5. This carrier structure 6′ protrudes away from the web 5 in the longitudinal direction L of the stent such that the second end in the longitudinal direction k of the carrier structure 6′ points in the direction of the basic mesh. In this way, this carrier structure 6′ is “framed” by the zigzag or meandering web 5 in the area of the stent wall, i.e., the meandering areas of the stent 5 surround the carrier structure 6′ on three sides in the area of the stent wall. Because of the attachment of the carrier structure 6, 6′ to the basic mesh 3 at their first finger-shaped end, the respective carrier structure 6, 6′ can be adapted flexibly to the movements of the zigzag or meandering web 5.

In one exemplary embodiment in FIG. 1b, two or more carrier structures 6″ protrude essentially away from the web 5 in the longitudinal direction L of the stent.

A functional element 8 having a spherical shape on the second end opposite the first end in the longitudinal direction is arranged on the carrier structures 6, 6′ on the second end opposite the first end in the longitudinal direction of the carrier structure 6, 6′. The functional element 8 surrounds the second end of the carrier structure 6, 6′ completely. In another exemplary embodiment, the functional element 8 may also have a disk shape whereby the circular cross section extends essentially in a plane running tangentially to the lateral surface of the cylindrical stent (tangential plane). The spherical functional element 8 completely surrounds the end of the carrier structure 6, 6′ protruding away from the basic mesh. In this way, the largest possible extent of the functional element in the direction of the tangential plane is achieved.

The functional element 8 may contain radiopaque material, preferably one or more of the radiopaque elements indicated above and/or one or more of the radiopaque compounds listed above. Examples of the material of the functional element are also listed above.

Additionally or alternatively, the functional element 8 may contain pharmaceutically active substances having an anti-inflammatory, antiproliferative and/or spasmolytic effect and consisting of, for example, the aforementioned group of active ingredients, which may be bonded to the carrier structure 6, 6′ with the help of a carrier matrix, preferably a polymer. After implantation of the stent, these active ingredients can elute into the body tissue and manifest their anti-inflammatory, antiproliferative and/or spasmolytic effects in the body tissue. In an especially preferred exemplary embodiment, the functional elements designed as an active ingredient depot are arranged so the functional elements are uniformly distributed over the entire wall of the mesh structure.

In one exemplary embodiment, the carrier structures 6, 6′ are made of an insulating material, e.g., a ceramic or a plastic.

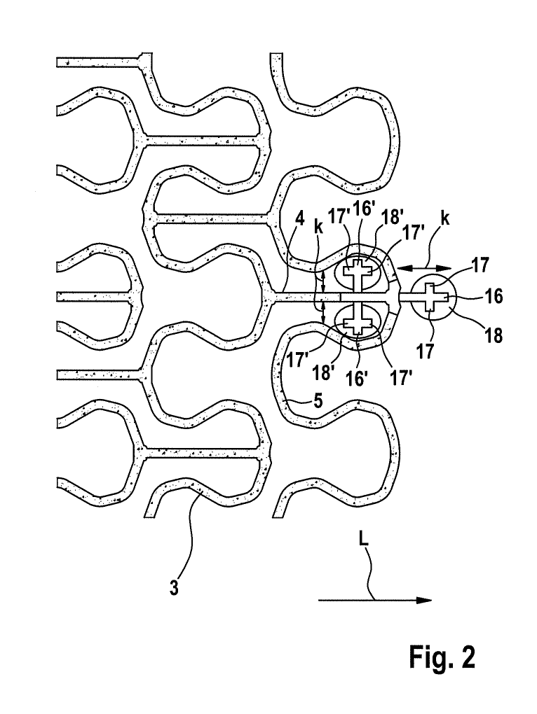

In the exemplary embodiment shown in FIG. 2, the carrier structures 16 are shown; they are designed to be finger shaped at the first end and are attached at the first end to the basic mesh 3 of the stent. On the second end, the carrier structures 16 each have two fingers 17 protruding laterally, i.e., perpendicular to the direction k. The carrier structure 16 thus forms a cross shape at the second end. The carrier structure 16 is connected to the zigzag or meandering web 5. Another carrier structure 16′, having a similar design, is connected at the first finger-shaped end to the web 4 of the basic mesh 3 running in the longitudinal direction L and protrudes away from the web 4 in the tangential plane essentially perpendicular to the longitudinal direction L.

In the case of the carrier structure 16, the functional element 18 arranged at the end of the carrier structure 16 and protruding away from the basic mesh 3 of the stent is designed to be essentially spherical or disk-shaped by analogy with the exemplary embodiment depicted in FIGS. 1a or 1b. The functional elements 18′ provided on the carrier structures 16′ are essentially droplet-shaped, each surrounding the second end of the carrier structure 16′ including the finger 17′ protruding laterally away at a right angle to the direction k.

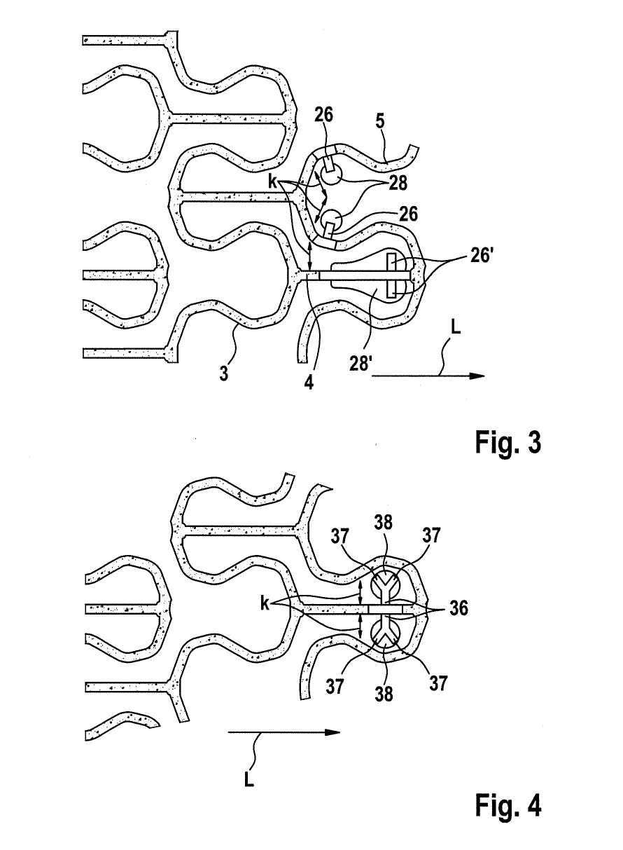

The carrier structure 26 depicted in FIG. 3 corresponds to the finger-shaped carrier structures 6, 6′ from FIG. 1a or 1b which are arranged on the zigzag or meandering webs 5 situated the greatest distance away in the direction of the end of the stent in the areas of these webs 5 which are curved inward in the direction of the basic mesh 3 (concave section). The zigzag or meandering webs 5 thus surround the carrier structures 6, and the functional elements 28 attach to the second end of the carrier structure 26 in the area of the stent wall on three sides. The functional elements 28 have a droplet shape here.

Another exemplary embodiment shown in FIG. 3 has two finger-shaped carrier structures 26′ arranged at opposite ends on a web 4 running in the longitudinal direction L in the tangential plane. The longitudinal direction k of the carrier structures 26′ runs perpendicular to the longitudinal direction L of the stent. The functional element 28′ in the droplet shape extends around the opposing carrier structures 26′ so that the functional element 28′ completely surrounds both carrier structures 26′ and, in addition, surrounds the nearest area of the web 4 running in the longitudinal direction to which the carrier structures 26′ are attached. This yields a particularly great extent of the functional element 28′ essentially in the longitudinal direction L of the stent.

By analogy with the functional element 28′, the droplet-shaped functional element 28 may also be extended in another exemplary embodiment to such an extent that it extends up to the respective web 5.

FIG. 4 shows another exemplary embodiment having a carrier structure 36 whereby the carrier structure 36 has essentially a Y shape in a longitudinal section. The carrier structure 36 is connected at the first finger-shaped end to a web 4 running in the longitudinal direction L. On the second end, the carrier structure 36 has two fingers 37 protruding away from one another at an acute angle. The functional element designed in a spherical or disk shape completely surrounds the second end of the carrier structure 36 having the fingers 37 protruding away from the basic mesh 3.

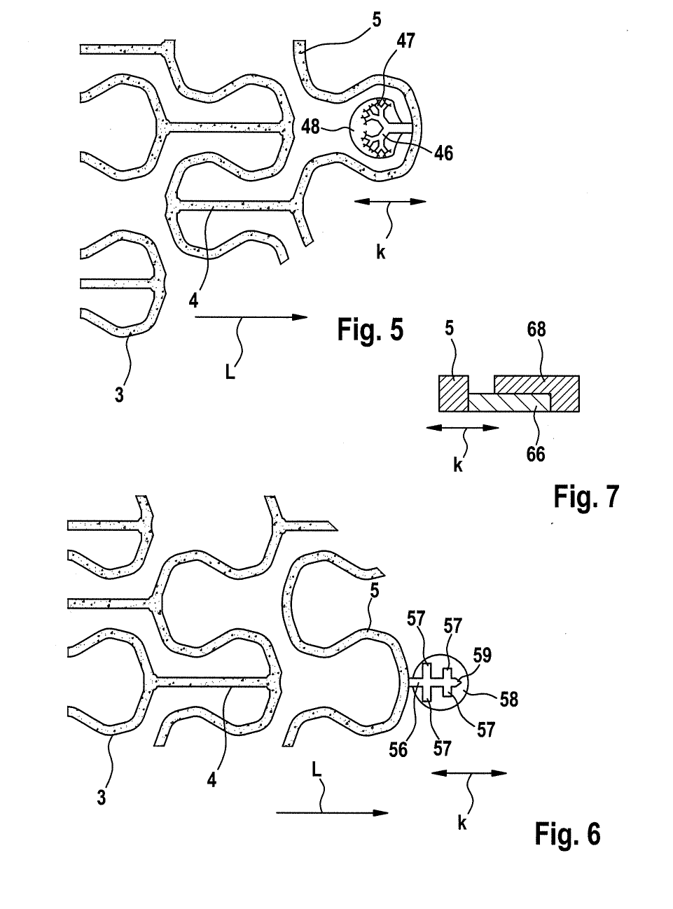

The exemplary embodiment illustrated in FIG. 5 has a carrier structure 46 which is designed on the first end that is connected to the web 5 of the stent. On the second end, protruding away from the basic mesh 3, the carrier structure 46 has fractal branches 47. The fractal branches are preferably provided in the plane of the stent wall. In other exemplary embodiments, branches may also be provided in a plane running radially with respect to the stent. The functional element 48 completely surrounds the second end of the carrier structure 46 with the fractal branches 47 in a droplet shape. This yields an especially tight anchoring of the functional element 48 on the carrier structure 46.

In another exemplary embodiment shown in FIG. 6, the carrier structure 56 has several fingers 57 arranged on the side of the second end, the length of the fingers in the longitudinal direction k being smaller in the direction of the second end of the carrier structure 56 so that essentially a tree structure is formed. The fingers 57 surround an area 59 of the carrier structure 56 representing the trunk, preferably around the entire circumference. By analogy with the previous exemplary embodiments, the functional element 58 surrounds the second end of the carrier structure 56 completely with the tree structure 57, 59.

In other exemplary embodiments, the carrier structure may have other shapes than those shown above on the second end, these shapes ensuring a good attachment of the functional element arranged on the second end and at least partially surrounding the carrier structure.

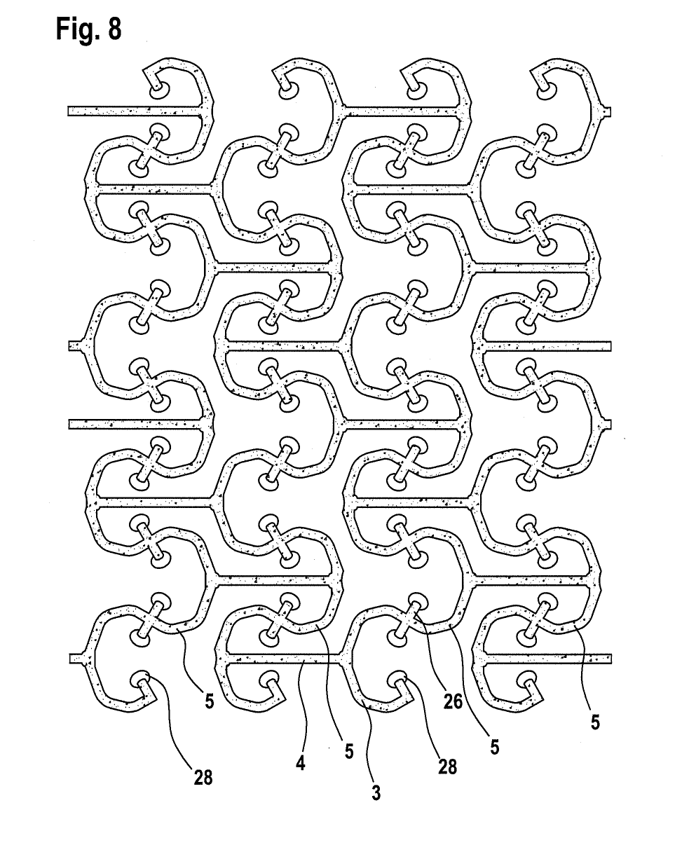

The exemplary embodiment shown in FIG. 7 has a finger-shaped carrier structure 66 by analogy with the structure shown in FIGS. 1a or 1b, which has a smaller thickness in the radial direction, based on the stent, then does the stent web 5. The functional element 68, which is essentially a disk shape, surrounds the carrier structure 66 on the second end, whereby the functional element 68 is arranged only on the top side of the carrier structure 66. This achieves the result that the total thickness of the element consisting of the carrier structure 66 and the functional element 68 is comparable to the thickness of the webs 4, 5 of the stent so that a large volume of the functional element 68 is achieved and a small influence of the flow of the liquids flowing in the container is also achieved.

On the whole, the stent may have the carrier structures with functional elements illustrated in FIGS. 1a through 6 distributed over the entire length, whereby approximately similarly shaped carrier structures and functional elements or differently shaped carrier structures and functional elements may be used. This exemplary variant is especially preferred when the functional elements contain pharmaceutically active substances. Such an exemplary embodiment is shown in FIG. 8. The functional elements 28 with the carrier structures 26 shown in FIG. 3 are arranged so the functional elements are distributed over the entire wall of the stent and opposite the meandering webs 5.

Alternatively, such carrier structures are arranged on the distal and/or proximal end of the endoprosthesis and especially preferably on a circumferential line.

All patents, patent applications and publications referenced herein are incorporated by reference herein in their entirety.

What is claimed is:

1. An endoprosthesis, in particular an intraluminal endoprosthesis, such as a stent, comprising:
(a) a basic mesh; and
(b) a functional element having a first end attached to a carrier structure and having a second end and having a different material composition in at least a portion of the volume of the functional element in comparison with the material of the basic mesh,
wherein the carrier structure is arranged on the basic mesh on the first essentially finger-shaped end and protrudes away from the basic mesh, and
wherein the carrier structure has a plurality of branches in the area of the second end, and
wherein the functional element is arranged on an area of the carrier structure that protrudes away from the base body and at least partially surrounds the area of the carrier structure.

2. The endoprosthesis of claim 1, wherein the functional element completely surrounds the second end of the carrier structure that protrudes away from the basic mesh.

3. The endoprosthesis of claim 1, wherein the functional element has a droplet shape, a disk shape or a spherical shape.

4. The endoprosthesis of claim 1, wherein the functional element consists at least partially of a radiopaque material.

5. The endoprosthesis of claim 4, wherein the radiopaque material comprises one or more of the elements selected from the group consisting of gold, platinum, silver, tungsten, iodine, tantalum, yttrium, niobium, molybdenum, ruthenium, rhodium, barium, lanthanum, cerium, praseodymium, neodymium, samarium, europium, gadolinium, terbium, dysprosium, holmium, erbium, thulium, ytterbium, lutetium, hafnium, rhenium, osmium and bismuth and one or more of the radiopaque compounds from these elements and barium sulfate, bismuth trioxide, bromine, iodine, iodide, titanium oxide and zirconium oxide.

6. The endoprosthesis of claim 1, wherein the functional element contains at least one pharmaceutically active substance.

7. The endoprosthesis of claim 6, wherein the at least one pharmaceutically active substance comprises one or more substances selected from the group of active ingredients consisting of calcium channel blockers, lipid regulators (e.g., fibrates), immunosuppressants, calcineurine inhibitors (e.g., tacrolimus), antiphlogistics (e.g., cortisone or diclofenac), anti-inflammatories (e.g., imidazoles), antiallergics, oligonucleotides (e.g., dODN), estrogens (e.g., genistein), endothelium-forming agents (e.g., fibrin), steroids, proteins/peptides and vasodilators (e.g., sartanes).

8. The endoprosthesis of claim 1, wherein the carrier structure is made of the same material as the basic mesh.

9. The endoprosthesis of claim 1, wherein the carrier structure is made at least partially of an electrically insulating material.

10. The endoprosthesis of claim 1, wherein the carrier structure is provided with an electrically insulating coating at least in the area in which the functional element is arranged on the carrier structure.

11. The endoprosthesis of claim 1, further comprising a plurality of carrier structures having functional elements containing a pharmaceutically active substance and which are arranged in uniform distribution over the entire wall of the mesh structure.

12. The endoprosthesis of claim 1, wherein the endoprosthesis has a plurality of carrier structures with functional elements with radiopaque material, which are arranged on the distal or proximal end of the endoprosthesis, preferably being arranged on a circumferential line.

13. The endoprosthesis of claim 6, wherein the pharmaceutically active substance has an anti-inflammatory, spasmolytic or antiproliferative effect.

14. The endoprosthesis of claim 14, wherein the electrically insulating material is plastic or ceramic.

15. An endoprosthesis, in particular an intraluminal endoprosthesis, such as a stent, comprising:
(a) a basic mesh; and
(b) a functional element having a first end attached to a carrier structure and having a second end and having a different material composition in at least a portion of its volume in comparison with the material of the basic mesh,
wherein the carrier structure is arranged on the basic mesh on the first essentially finger-shaped end and protrudes away from the basic mesh,
wherein the carrier structure has a plurality of fractal branches proximate to the second end, and
wherein the functional element is arranged on an area of the carrier structure that protrudes away from the base body and at least partially surrounds the area of the carrier structure.

16. An endoprosthesis, in particular an intraluminal endoprosthesis, such as a stent, comprising:
(a) a generally cylindrical shaped mesh structure made of a first material and having at least one finger-shaped first end and a second end, the second end having a plurality of fractal branches proximate thereto;
(b) a carrier structure arranged on the basic mesh on the first essentially finger-shaped end and protruding away from the mesh structure; and,
(c) a functional element attached to the carrier structure and proximate to an area of the carrier structure that protrudes away from the mesh structure and at least partially surrounds the area of the carrier structure, at least a portion of the volume of the functional element being made of a material different than the first material.
